(19) United States
(12) Patent Application Publication
(10) Pub. No.: US 2010/0298549 A1
(43) Pub. Date: Nov. 25, 2010

(54) Extraction method for target bio-molecule from a sample solution and fabrication of extraction module

(75) Inventor: Chien-Kuang Chen, Taipei (TW)

Correspondence Address: J C PATENTS, 4 VENTURE, SUITE 250, IRVINE, CA, 92618, US

(73) Assignee: National Taiwan University of Science and Technology, Taipei (TW)

(21) Appl. No.: 12/454,686

(22) Filed: May 20, 2009

(51) Int. Cl. C07K 1/34; C07H 1/06; B01D 15/00; B05D 3/00; C08F 2/46

(52) U.S. Cl. 530/427; 536/25.41; 422/101; 427/307; 427/488; 427/539

(57) ABSTRACT

An extraction method for a target bio-molecule of a sample solution is described. First, a substrate having a thermally responsive polymer brush immobilized thereon is provided. The thermally responsive polymer brush has a lower critical solution temperature (LCST). Thereafter, while the sample solution flows over the substrate, the temperature of the sample solution or the substrate or both is decreased to less than the LCST, so that the target bio-molecule of the sample solution is captured inside the thermally responsive polymer brush. Afterwards, while the extraction solution flows over the substrate, the temperature of the extraction solution or the substrate or both is increased to more than the LCST, so that the captured target bio-molecule of the sample solution is released out of the thermally responsive polymer brush.

### BACKGROUND OF THE INVENTION

1. Field of Invention

The present invention relates to a lab-on-a-chip (LOC), and more generally to extraction method for a bio-molecule from a sample solution and fabrication of the extraction module.

2. Description of Related Art

Generally, a lab-on-a-chip (LOC) is a device that integrates multiple laboratory functions on a single chip, and the dimension thereof is about several millimeters or centimeters. The LOC can detect and analyze an extremely small fluid volume down to a pico-liter quickly and correctly.

In recently years, gene therapy is gradually applied to cure gene deficiencies or life-threatening diseases and makes a breakthrough in medical treatment for many gene or immune related diseases. Generally, DNA detection includes three main parts: DNA extraction module, polymerase chain reaction (PCR) process for DNA signal amplification and DNA hybridization module. The LOC for the DNA detection is designed to integrate the three main parts into a micro-fluidic system, so as to automate the DNA sample detection.

The PCR amplification process and the DNA hybridization module have been widely studied, but the DNA extraction module still has room for development. Accordingly, how to effectively extract the target DNA so as to speed up the DNA detection has been one of the main topics in the industry.

### SUMMARY OF THE INVENTION

The present invention provides an extraction method, in which the nano-extraction of a target bio-molecule suspended in a sample solution can be easily achieved.

The present invention further provides an extraction module and a fabrication method thereof. The fabricated extraction module is beneficial to enhance the extraction efficiency and speed up the DNA detection.

The present invention provides an extraction method for a target bio-molecule from a sample solution. First, a substrate having a thermally responsive polymer brush immobilized thereon is provided. The thermally responsive polymer brush has a lower critical solution temperature (LCST). Thereafter, while the sample solution flows over the substrate, the temperature of the sample solution or the substrate or both is decreased to less than the LCST, so that the target bio-molecule of the sample solution is captured inside the thermally responsive polymer brush. Afterwards, while the extraction solution flows over the substrate, the temperature of the extraction solution or the substrate or both is increased to more than the LCST, so that the captured target bio-molecule of the sample solution is released out of the thermally responsive polymer brush.

According to an embodiment of the present invention, the substrate includes a silicon substrate or a glass substrate, for example.

According to an embodiment of the present invention, the thermally responsive polymer brush includes poly(N-isopropylacrylamide) or a copolymer of N-isopropylacrylamide and acrylic acid or acylic amide, for example.

According to an embodiment of the present invention, the thermally responsive polymer brush includes a plurality of thermally responsive polymer chains, and at least a portion of the thermally responsive polymer chains have a length of more than about 50 nm, for example.

According to an embodiment of the present invention, the extraction solution includes water, for example.

According to an embodiment of the present invention, the target bio-molecule is positively charged, negatively charged or electrically neutral.

According to an embodiment of the present invention, the target bio-molecule includes DNA, RNA, protein, fluorescein isothiocyanate (FITC), adenosine triphosphate (ATP), c-myc antisense oligonucleotide (As-myc), for example.

According to an embodiment of the present invention, the sample solution flows over the substrate with backward and forward cycles.

The present invention further provides an extraction module for a target bio-molecule of a sample solution. The extraction module includes a substrate and a thermally responsive polymer brush. The substrate has at least one groove. The thermally responsive polymer brush is immobilized on the surface of the groove and the substrate.

According to an embodiment of the present invention, the substrate includes a silicon substrate or a glass substrate, for example.

According to an embodiment of the present invention, the thermally responsive polymer brush includes poly(N-isopropylacrylamide) or a copolymer of N-isopropylacrylamide and acrylic acid or acylic amide, for example.

According to an embodiment of the present invention, the thermally responsive polymer brush includes a plurality of thermally responsive polymer chains, and at least a portion of the thermally responsive polymer chains have a length of more than about 50 nm, for example.

According to an embodiment of the present invention, the target bio-molecule is positively charged, negatively charged or electrically neutral.

According to an embodiment of the present invention, the target bio-molecule includes DNA, RNA, protein, FIFC, ATP or As-myc, for example.

According to an embodiment of the present invention, the extraction module further includes a plurality of protruding parts extending from the bottom of the groove.

According to an embodiment of the present invention, the extraction module further includes a sample injection member disposed on the substrate and configured to connect the groove.

According to an embodiment of the present invention, the sample injection member includes a body, two sample injection inlets and a connection part. The two sample injection inlets are respectively disposed beside the body. The connection part is configured to connect the body and the groove.

The present invention further provides a method of fabricating an extraction module for a bio-molecular from a sample solution. First, a substrate is provided. Thereafter, at least one groove is formed in the substrate. Afterwards, a thermally responsive polymer brush is immobilized on the surface of the groove and the substrate.

According to an embodiment of the present invention, the thermally responsive polymer brush is grafted on the substrate by using atom transfer radical polymerization through oxygen plasma treatment.

According to an embodiment of the present invention, the method of fabricating the extraction module further includes performing an oxide plasma treatment on the substrate to increase hydrophilicity or polarity of the substrate before the step of grafting the thermally responsive polymer brush on the substrate.

In view of above, the extraction method in accordance with the present invention can be applied to identify a target bio-molecule suspended in a sample solution. The target bio-molecule is captured in the thermally responsive polymer brush by decreasing the temperature of the sample solution or the substrate or both to less than the LCST of the polymer brush, and the target bio-molecule is released out of the thermally responsive polymer brush by increasing the temperature of the extraction solution or the substrate or both to more than the LCST of the polymer brush. The extraction method is simple, fast, accurate and low-cost, so that the competitive advantage is easily achieved.

Further, in the extraction module of the present invention, a thermally responsive polymer brush is immobilized on a substrate. Due to the temperature-sensitive property of the thermally responsive polymer brush, the extraction module of the present invention can be used for extracting a target bio-molecule of a sample solution effectively.

In order to make the aforementioned and other objects, features and advantages of the present invention comprehensible, a preferred embodiment accompanied with figures is described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1A schematically illustrates a top view of an extraction module for a target bio-molecule of a sample solution according to an embodiment of the present invention;

FIG. 1B schematically illustrates a cross-sectional view taken along the line A-A of FIG. 1A.

FIGS. 2A to 2C schematically illustrate an extraction method for a target bio-molecule of a sample solution according to an embodiment of the present invention.

FIGS. 3A to 3D illustrate a synthesis process of a thermally responsive polymer brush on a substrate according to Example 1 of the present invention.

FIG. 4 represents an atom force microscopy image for a substrate with a thermally responsive polymer brush thereon at 20° C. according to Example 1 of the present invention.

FIG. 5 represents an agarose gel electrophoresis diagram of the extracted target DNA molecule after a PCR amplification process.

### DESCRIPTION OF EMBODIMENTS

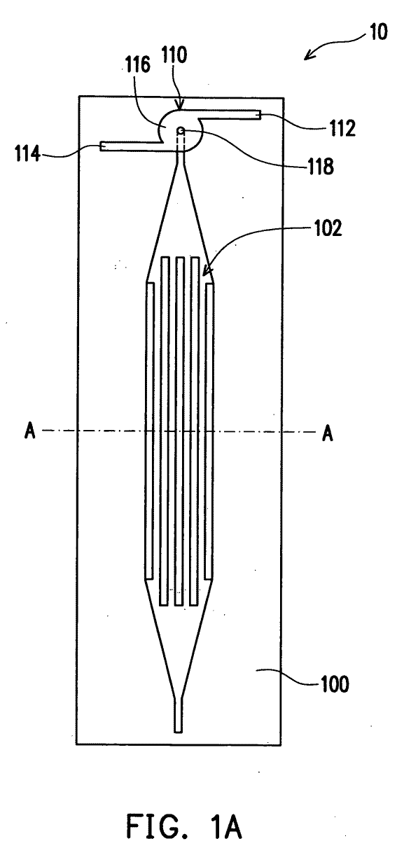

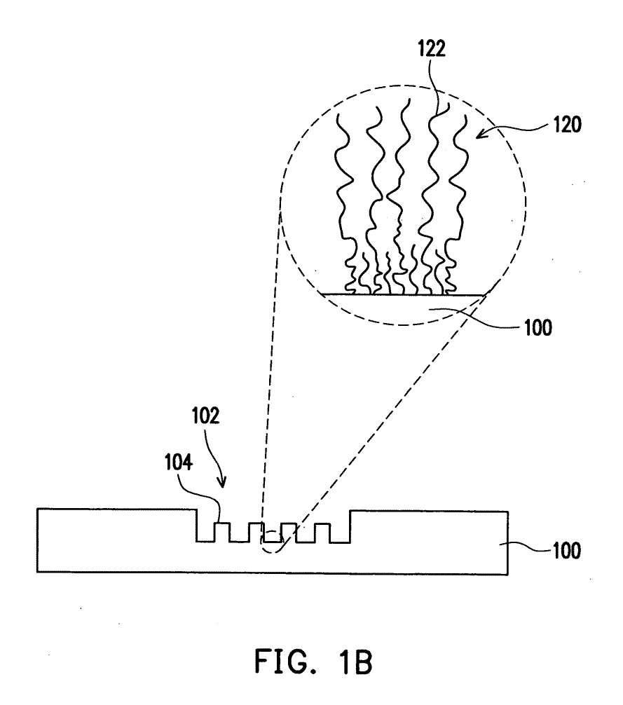

FIG. 1A schematically illustrates a top view of an extraction module for a target bio-molecule of a sample solution according to an embodiment of the present invention.

FIG. 1B schematically illustrates a cross-sectional view taken along the line A-A of FIG. 1A.

Referring to FIG. 1A and 1B, the extraction module 10 includes a substrate 100 and a thermally responsive polymer brush 120. The substrate 100 includes a silicon substrate or a glass substrate, for example. The substrate 100 has at least one groove 102 therein as a fluid channel for the sample solution. The method of forming the groove 102 includes using a hot embossing process, for example. The thermally responsive polymer brush 120 is immobilized on the surface of the groove 102 and the substrate 100. As shown in FIG. 1B, a plurality of protruding parts 104 are extended from the bottom of the groove 102, so as to increase the reaction area between the sample solution and the thermally responsive polymer brush 120, thereby enhancing the extraction efficiency of the target bio-molecule of the sample solution.

The thermally responsive polymer brush 120 includes a plurality of thermally responsive polymer chains 122, and the material thereof includes poly(N-isopropylacrylamide) (PNIPAAm) or a copolymer of N-isopropylacrylamide (NIPAAm) and acrylic acid or acylic amide, for example. In an embodiment, the thermally responsive polymer brush 120 is grafted on the substrate 100 by using atom transfer radical polymerization (ATRP), for example. The thermally responsive polymer brush 120 is bonded to the substrate 100 by the strong hydrogen bonding, for example. Before the step of grafting the thermally responsive polymer brush 120 on the substrate 100, an oxide plasma treatment can be optionally performed on the substrate 100, so as to increase hydrophilicity or polarity of the substrate 100. In details, the oxide plasma treatment can increase hydroxyl groups on the surface of the substrate 100, so as to increase the density of the thermally responsive polymer chains 122 which are subsequently formed.

The thermally responsive polymer brush 120 exhibits a temperature-sensitive property, and causes a reversibly physical change as the temperature is varied. That is, the thermally responsive polymer brush 120 has a lower critical solution temperature (LCST). The thermally responsive polymer brush 120 physically swells below its LCST and shrinks above its LCST. In other words, each thermally physical polymer chain 122 is like an extended coil below its LCST, and changes to a globule above its LCST.

In an embodiment, the thermally responsive polymer brush 120 includes PNIPAAm, and the LCST thereof is from about 30 to 33° C., for example. Acrylic acid or acylic amide can be optionally added to NIPAAm during the step of grafting the thermally responsive polymer brush 120 on the substrate 100, so as to adjust the LCST to be lower than 30° C. or higher than 33° C.

The temperature-sensitive property of the thermally responsive polymer brush 120 can be used for extracting the target bio-molecule of the sample solution. In details, when the temperature of the sample solution or the substrate 100 or both is decreased to less than the LCST, the thermally responsive polymer brush 120 swells and renders hydrophilic property to capture the target bio-molecule inside the thermally responsive polymer brush 120. When the temperature of the extraction solution or the substrate 100 or both is increased to more than the LCST, the thermally responsive polymer brush 120 shrinks and renders hydrophobic property to release the target bio-molecule out of the thermally responsive polymer brush 120.

Referring to FIG. 1A, the extraction module 10 further includes a sample injection member 110. The sample injection member 110 is disposed on the substrate 100 and configured to connect to the groove 102. The sample injection member 110 includes sample injection inlets 112 and 114, a body 116 and a connection part 118. The sample injection member 110 is a helix-like structure, for example. The sample injection inlets 112 and 114 are respectively disposed beside the body 116. The connection part 118 connects the body 116 and the groove 102.

The sample solution is injected to the extraction module 10 through the sample injection inlets 112 and 114 by an air micro-pump. The air micro-pump controls the fluidic velocity and direction of the sample solution. The sample solution is fully mixed in the body 116, and the mixed solution is then injected to the groove 102 through the connection part 118. Thereafter, the target bio-molecule suspended in the sample solution is extracted by the thermally responsive polymer brush 120, and the extraction mechanism is described in the following.

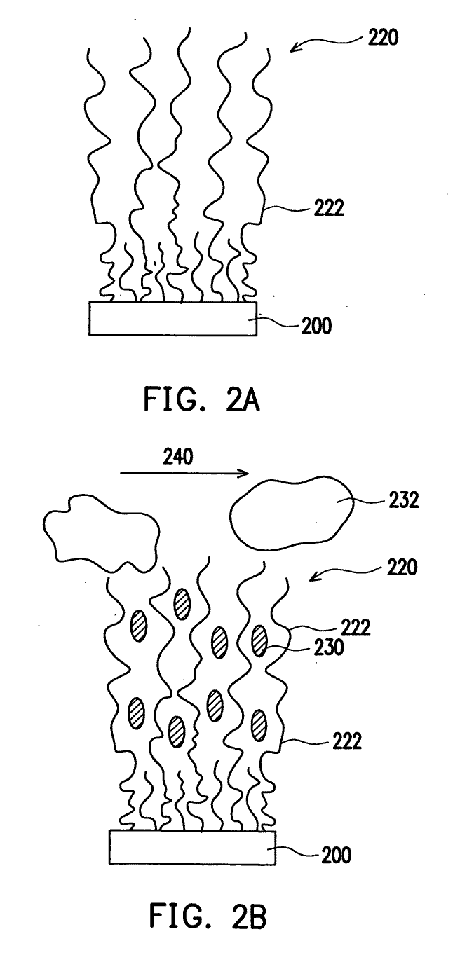

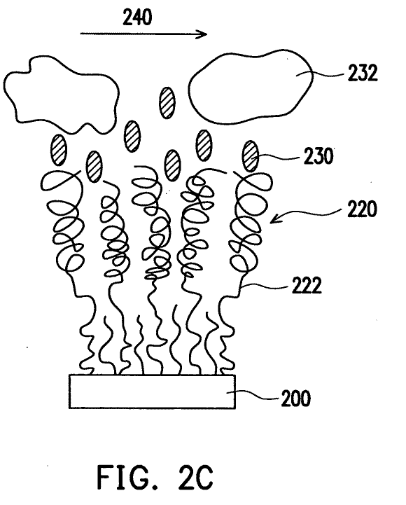

FIGS. 2A to 2C schematically illustrate an extraction method for a target bio-molecule of a sample solution according to an embodiment of the present invention. The extraction method is conducted in a fluidic system. In an embodiment, the extraction method is conducted in the extraction module as described in FIGS. 1A to 1B, for example.

Referring to FIGS. 2A, a substrate 200 having a thermally responsive polymer brush 220 immobilized thereon is provided. The substrate 200 is a silicon substrate or a glass substrate, for example. The thermally responsive polymer brush 220 has a LCST. The thermally responsive polymer brush 220 includes PNIPAAm or a copolymer of NIPAAm and acrylic acid or acylic amide, for example. In an embodiment, the thermally responsive polymer brush 220 includes PNIPAAm, and the LCST thereof is between about 30 to 33° C., for example. Further, the thermally responsive polymer brush 220 includes a plurality of thermally responsive polymer chains 222, and at least a portion of the thermally responsive polymer chains 222 have a length of more than about 50 nm, for example. Preferably, at least a portion of the thermally responsive chains 222 have a length of more than about 70 nm, for example.

Referring to FIG. 2B, the sample solution including a target bio-molecule 230, a human blood cell and a buffer solution is prepared. The human blood cell in the sample solution is designed as a background bio-macromolecule 232 for the detection of the target bio-molecule 230. In an embodiment, the target bio-molecule 230 can be a DNA molecule of Escherichia coli (E. Coli), severe acute respiratory syndrome (SARS) or acquired immune deficiency syndrome (AIDS), for example. In another embodiment, the target bio-molecule 230 can be RNA, protein, fluorescein isothiocyanate (FITC), adenosine triphosphate (ATP), c-myc antisense oligonucleotide (As-myc) or the like, for example. Further, the target bio-molecule can be positively charged, negatively charged or electrically neutral.

The sample solution flows over the surface of the substrate 200 along the direction 240. The temperature of the sample solution or the substrate 200 or both is decreased to less than the LCST of the thermally responsive polymer brush 220, such as 20° C., for example. When the temperature of the sample solution or the substrate 200 or both is maintained at about 20° C., the inter-molecular hydrogen bonding is generated between the target bio-molecule 230 and each thermally responsive polymer chain 222. Therefore, the target bio-molecule 230 suspended in the sample solution is captured inside the thermally responsive polymer brush 220.

The sample solution flows over the surface of the substrate 200 by an air micro-pump for controlling its fluidic velocity and direction. In an embodiment, the sample solution flows over the surface of the substrate 200 with backward and forward cycles for about 30 to 180 seconds, for example. That is, the sample solution with a temperature of below LCST flows over the surface of the substrate 200 along the direction 240, and then flows over the same along the direction opposite to the direction 240, so as to increase the efficiency of capturing the target bio-molecule 230. Accordingly, the concentration of the target bio-molecule 230 of the sample solution can be extremely small, and even the nano-extraction can be easily achieved by flowing the sample solution over the substrate 200 with backward and forward cycles. Thereafter, the unexpected bio-macromolecule 232 is removed by using a buffer solution to clean the surface of the substrate 200.

Referring to FIG. 2C, the extraction solution flows over the surface of the substrate 200 along the direction 240. The extraction solution includes water or the like, for example. The temperature of the extraction solution or the substrate 200 or both is increased to more than the LCST of the thermally responsive polymer brush 220, such as 40° C., for example. When the temperature of the extraction solution or the substrate 200 or both is maintained at about 40° C., the inter-molecular hydrogen bonding generated between the target bio-molecule 230 and each thermally responsive polymer chain 222 is broken, and each thermally responsive polymer chain 222 shrinks and tends to generate the intra-molecular hydrogen bonding with itself. Accordingly, the target bio-molecule 230 is released out of the thermally responsive polymer brush 220. Thereafter, a PCR process is performed to amplify the signal of the target bio-molecule 230.

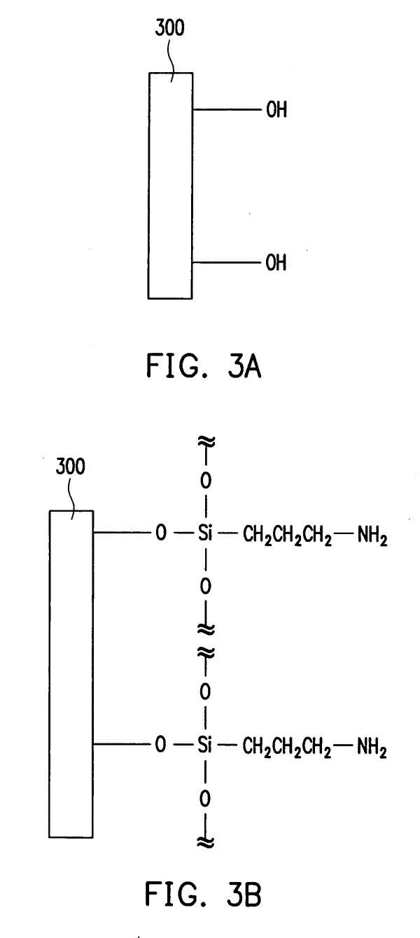

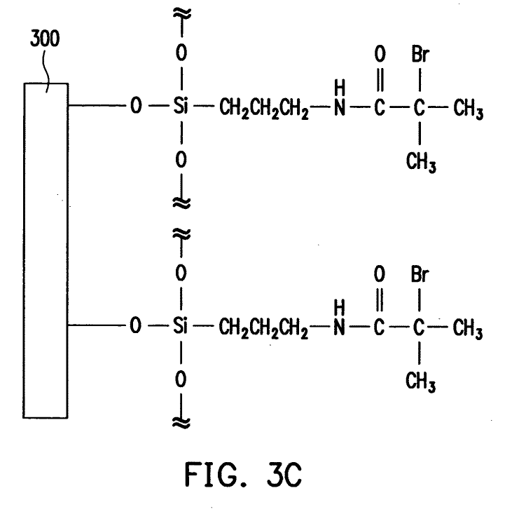

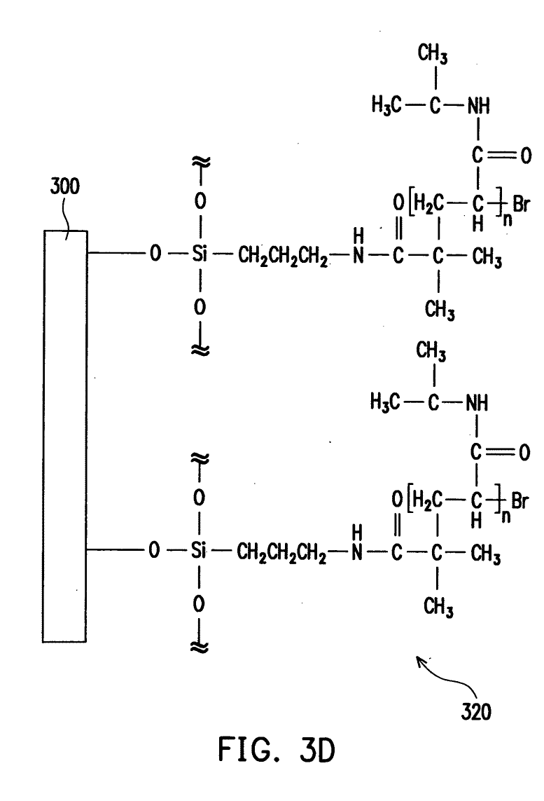

Example 1 is provided to prove the performance of the extraction method of the present invention. FIGS. 3A to 3D illustrate a synthesis process of a thermally responsive polymer brush on a substrate. Example 1 in which the thermally responsive polymer brush includes PNIPAAm and the target bio-molecule is a DNA molecule of E. Coli is provided for illustration purposes, and is not construed as limiting the present invention. It is appreciated by persons skilled in the art that the thermally responsive polymer brush can include a copolymer of NIPAAm and acrylic acid or acrylic amide, and the target bio-molecule can be RNA, protein, FITC, ATP, As-myc or the like.

### EXAMPLE 1

Referring to FIG. 3A, an oxygen plasma treatment was performed on the substrate 300, so as to increase hydroxyl groups on the substrate 300. The substrate 300 was a silicon substrate. The parameters of the oxide plasma treatment included a pressure of about 0.03 Torr, a RF power of about 300 W, a gas source of about 50% argon and about 50% oxygen and a time of about 10-60 seconds, for example.

Referring to FIG. 3B, a mixed solution of sulfuric acid and hydrogen peroxide was used to modify the surface of the substrate 300. Thereafter, 0.5 ml of 3-aminopropyltriethoxy silane (APTES) was dissolved in 5 ml of toluene to prepare a solution, and the substrate 300 was placed in the solution for reaction at room temperature for 2 hours. Afterwards, toluene and ethanol were used to clean the surface of the substrate 300. At this step, a self-assembled monolayer (SAM) was formed on the surface of the substrate 300.

Referring to FIG. 3C, 0.5 ml of α-Bromoisobutyl bromide was dissolved in 0.5 ml of triethylamine and 25 ml of tetrahydrofuran (THF) to prepare a mixed solution. Thereafter, the substrate 300 was placed in the mixed solution for reaction at 0° C. for 1 hour and then at room temperature for 12 hours. Afterwards, THF and ethanol were used to clean the surface of the substrate 300. At this step, initiators for polymer grafting were formed on the surface of the substrate 300.

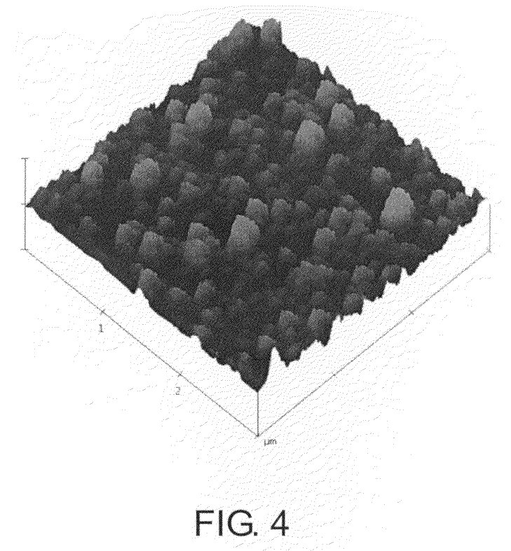

Referring to FIG. 3D, the substrate 300 was placed in a flask under nitrogen atmosphere. Thereafter, 0.027 g (0.0185 mmole) of Copper(I) bromide (CuBr), 0.0117 ml (0.557 mmole) of n,n,n′,n′,n-Pentamethyldiethylenetriamine (PMDETH), 2.1 g (18.3 mmole) of NIPAAm, 10 ml of methanol and 10 ml of water were added to the flask, and reacted with the substrate 300 at room temperature for 2 hours. Afterwards, methanol and water were used to clean the surface of the substrate 300. At this step, atom transfer radical polymerization was carried out to graft PNIPAAm on the substrate 300, and at least a portion of the thermally responsive polymer chains have a length of about 71 nm. Accordingly, a thermally responsive polymer brush 320 was grafted on the surface of the substrate 300, and the roughness of the substrate 300 at 20° C. was up to 10.582 nm, as shown in FIG. 4. The thermally responsive polymer brush 320 rendered hydrophilic property below its LCST of 30-33° C. (e.g. 20° C.), causing the value of contact angle for water closed to 600. On the other hand, the thermally responsive polymer brush 320 rendered hydrophobic property above its LCST of 30-33° C. (e.g. 40° C.), causing the value of contact angle for water closed to 80°.

After the substrate 300 having the thermally responsive polymer brush 320 immobilized thereon was synthesized, the extraction method as described in FIG. 2A to 2C was performed. The target bio-molecule was a DNA molecule of E. Coli and the concentration thereof is about 2×10⁻³ g/l, for example.

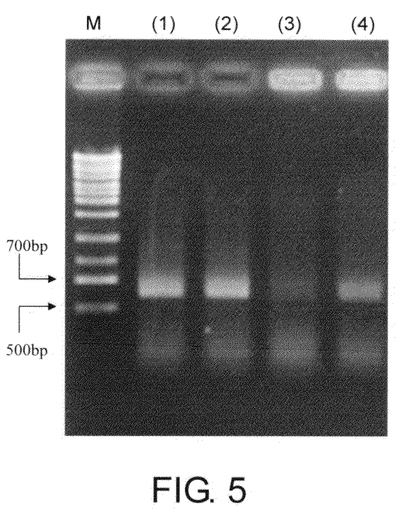

In Example 1, the encapsulated bio-macromolecule lysed from the human blood cell and the target DNA molecule of E. coli were released out of the thermally responsive polymer brush 320. Thereafter, an PCR amplification process is performed to enhance the quantity of the target DNA molecule for identification. The existence for the target DNA molecule of E. coli was confirmed through the PCR amplification process by the specific primer. An agarose gel electrophoresis diagram of the target DNA molecule of E. coli was shown in FIG. 5.

Referring to FIG. 5, lanes (1) represented the control group to confirm the gel electrophoresist process. Lanes (2) to (4) represented the experimental groups under the conditions that the sample solution temperature was at 25° C. (below LCST) and the extraction solution temperature were at 50° C. (above LCST), 25° C. (below LCST) and 33° C. (around LCST) respectively. The lanes (1) to (4) demonstrated the same result of 620 base pair (bp) amplified by PCR. The same position of 620 bp in the control group and the experimental groups proved that the target DNA molecule of E. coli cell was extracted by the extraction method of the present invention. Further, the lane (2) had greater brightness than lanes (3) and (4). It proved that higher temperature of the extraction solution helped to release more target DNA molecules.

In summary, the extraction module of the present invention includes a thermally responsive polymer brush on a substrate. The temperature-sensitive property of the thermally responsive polymer brush can be used for extracting a target bio-molecule of a sample solution. The manufacturing process of the extraction module is simple and the fabrication cost of the same is low.

Further, the extraction method of the present invention is conducted in a fluidic system having a substrate with a thermally responsive polymer brush thereon. The target bio-molecule suspended in the sample solution can be easily extracted by decreasing and increasing the temperature of the solution or the substrate or both. The target bio-molecule is captured in the thermally responsive polymer brush by decreasing the temperature of the injected sample solution or the substrate or both to less than the LCST of the polymer brush, and the target bio-molecule is released out of the thermally responsive polymer brush by increasing the temperature of the injected extraction solution or the substrate or both to more than the LCST of the polymer brush. The extraction method is simple, fast, accurate and low-cost, so that the competitive advantage is easily achieved.

Moreover, the present invention can be integrated with other laboratory functions on a single lab-on-a-chip, and parallel testing can be conducted on the sample solution including various bio-molecules. A specific DNA molecule suspended in the sample solution can be easily identified with one droplet of human blood, and complicated procedure and expensive system are not required.

This invention has been disclosed above in the preferred embodiments, but is not limited to those. It is known to persons skilled in the art that some modifications and innovations may be made without departing from the spirit and scope of this invention. Hence, the scope of this invention should be defined by the following claims.

What is claimed is:

1. An extraction method for a target bio-molecule from a sample solution, comprising:
providing a substrate having a thermally responsive polymer brush immobilized thereon, wherein the thermally responsive polymer brush has a lower critical solution temperature (LCST);
flowing the sample solution over the substrate and decreasing a temperature of the sample solution or the substrate or both to less than the LCST, so that the target bio-molecule of the sample solution is captured inside the thermally responsive polymer brush; and
flowing extraction solution over the substrate and increasing a temperature of the extraction solution or the substrate or both to more than the LCST, so that the captured target bio-molecule of the sample solution is released out of the thermally responsive polymer brush.

2. The extraction method of claim 1, wherein the substrate comprises a silicon substrate or a glass substrate.

3. The extraction method of claim 1, wherein the thermally responsive polymer brush comprises poly(N-isopropylacrylamide) or a copolymer of N-isopropylacrylamide and acrylic acid or acylic amide.

4. The extraction method of claim 1, wherein the thermally responsive polymer brush comprises a plurality of thermally responsive polymer chains, and at least a portion of the thermally responsive polymer chains have a length of more than about 50 nm.

5. The extraction method of claim 1, the extraction solution comprises water.

6. The extraction method of claim 1, wherein the target bio-molecule is positively charged, negatively charged or electrically neutral.

7. The extraction method of claim 1, wherein the target bio-molecule comprises DNA, RNA, protein, FIFC, ATP or As-myc.

8. The extraction method of claim 1, wherein the sample solution flows over the substrate with backward and forward cycles.

9. An extraction module for a target bio-molecule of a sample solution, comprising:
a substrate, having at least one groove; and
a thermally responsive polymer brush, immobilized on a surface of the groove and the substrate.

10. The extraction module of claim 9, wherein the substrate comprises a silicon substrate or a glass substrate.

11. The extraction module of claim 9, wherein the thermally responsive polymer brush comprises poly(N-isopropylacrylamide) or a copolymer of N-isopropylacrylamide and acrylic acid or acylic amide.

12. The extraction module of claim 9, wherein the thermally responsive polymer brush comprises a plurality of thermally responsive polymer chains, and at least a portion of the thermally responsive polymer chains have a length of more than about 50 nm.

13. The extraction module of claim 9, wherein the target bio-molecule is positively charged, negatively charged or electrically neutral.

14. The extraction module of claim 9, wherein the target bio-molecule comprises DNA, RNA, protein, FIFC, ATP or As-myc.

15. The extraction module of claim 9, further comprising a plurality of protruding parts extending from a bottom of the groove.

16. The extraction module of claim 9, further comprising a sample injection member disposed on the substrate and configured to connect the groove.

17. The extraction module of claim 16, wherein the sample injection member comprises:
a body;
two sample injection inlets, respectively disposed beside the body; and
a connection part, configured to connect the body and the groove.

18. A method of fabricating an extraction module for a bio-molecular from a sample solution, comprising:
providing a substrate;
forming at least one groove in the substrate; and
immobilize a thermally responsive polymer brush on a surface of the groove and the substrate.

19. The method of claim 18, wherein the thermally responsive polymer brush is grafted on the substrate by using atom transfer radical polymerization through oxygen plasma treatment.

20. The method of claim 18, further comprising performing an oxide plasma treatment on the substrate to increase hydrophilicity or polarity of the substrate before the step of grafting the thermally responsive polymer brush on the substrate.
